(19) United States
(12) Patent Application Publication
(10) Pub. No.: US 2025/0288251 A1
(43) Pub. Date: Sep. 18, 2025

(54) DEVICE FOR ENSURING ACCURATE AND PRECISE PLACEMENT OF A MEDICAL SENSOR ON A PATIENT'S CHEST

(71) Applicants: OHIO STATE INNOVATION FOUNDATION, Columbus, OH (US); TEGOSENS, INC., Columbus, OH (US)

(72) Inventors: Abraham Joseph, Dublin, OH (US); William T. Abraham, Powell, OH (US); Emre Ertin, Lewis Center, OH (US); Joseph Juratovac, Columbus, OH (US); Jeff Debord, Worthington, OH (US); Delano Ellis, Gahanna, OH (US); Maria Basile, Columbus, OH (US)

(73) Assignees: OHIO STATE INNOVATION FOUNDATION, Columbus, OH (US); TEGOSENS, INC., Columbus, OH (US)

(21) Appl. No.: 18/860,354

(22) PCT Filed: Apr. 25, 2023
(86) PCT No.: PCT/US2023/066157

§ 371 Date: Oct. 25, 2024

Related U.S. Application Data
(60) Provisional application No. 63/334,560, filed on Apr. 25, 2022.

(51) Int. Cl. A61B 5/00

(52) U.S. Cl. CPC A61B 5/6841; A61B 5/6823; A61B 5/6833

(57) ABSTRACT

A device for determining a precise placement of a medical sensor on a patient's chest is provided, the device comprising: a vertical portion including a sternal notice index portion for alignment with the patient's sternal notch, wherein the sternal notch index portion extends along a portion of the patient's sternum, wherein the vertical portion includes vertical patient size indices; and an arm extending from the vertical portion across the patient's midclavicular line, wherein the arm is oriented at a right angle to the vertical portion, wherein the arm includes horizontal patient size indices.

### CROSS-REFERENCE TO RELATED APPLICATIONS

This application claims priority from U.S. Provisional Patent Application No. 63/334,560, filed on Apr. 25, 2022, which is incorporated by reference herein in its entirety.

### BACKGROUND

Placing a medical sensor in a specific optimal position for use is difficult, particularly for patients, caregivers, or undertrained medical personnel. What is needed is a device to determine optimal placement of a medical sensor via reference to specific anatomical features of a patient. What is also needed is a base for ensuring repeatable optimal placement of a medical sensor on a patient's body.

### SUMMARY

In one aspect, a device for determining a precise placement of a medical sensor on a patient's chest is provided, the device comprising: a vertical portion including a sternal notice index portion for alignment with the patient's sternal notch, wherein the sternal notch index portion extends along a portion of the patient's sternum, wherein the vertical portion includes vertical patient size indices; and an arm extending from the vertical portion across the patient's midclavicular line, wherein the arm is oriented at a right angle to the vertical portion, wherein the arm includes horizontal patient size indices.

In another aspect, a system for mounting a base and a medical sensor on a patient's body is provided, the system comprising: a medical sensor including a retention element, wherein the retention element includes at least one retention slot; and a base including at least one base connector having an upper tab portion, wherein the upper tab portion of the at least one base connector engages the at least one retention slot.

In another aspect, a system for mounting a base and a medical sensor on a patient's body is provided, the system comprising: a medical sensor including a retention element, wherein the retention element includes at least one retention tab; and a base including at least one base connector having a slot and a cutout, wherein the slot of the at least one base connector engages the at least one retention tab.

### BRIEF DESCRIPTION OF THE FIGURES

The accompanying figures, which are incorporated in and constitute a part of the specification, illustrate various example aspects, and are used merely to illustrate various example aspects. In the figures, like elements bear like reference numerals.

FIG. 1 illustrates a device 100 for ensuring accurate and precise placement of a medical sensor on a patient's chest.

FIG. 2 illustrates a device 200 for ensuring accurate and precise placement of a medical sensor on a patient's chest.

FIG. 3 illustrates a device 300 for ensuring accurate and precise placement of a medical sensor on a patient's chest.

FIG. 4 illustrates a base 416 for receiving a medical sensor 430.

FIG. 5 illustrates a base 516 for receiving a medical sensor 530 placed on a patient's chest 532.

FIG. 6 illustrates a base 616 for receiving a medical sensor 630.

FIG. 7 illustrates a base 716 for receiving a medical sensor 730 placed on a patient's chest 732.

FIG. 8 illustrates a device 800 for ensuring accurate and precise placement of a medical sensor on a patient's chest.

FIG. 9 illustrates a device 900 for ensuring accurate and precise placement of a medical sensor on a patient's chest.

FIG. 10 illustrates a device 1000 for ensuring accurate and precise placement of a medical sensor on a patient's chest.

FIG. 11 illustrates a device 1100 for ensuring accurate and precise placement of a medical sensor on a patient's chest.

FIG. 12 illustrates using one's hand 1260 for ensuring accurate and precise placement of a medical sensor on a patient's chest.

FIG. 13A illustrates a perspective view of a mounting system 1300, including a base 1316 for receiving a medical sensor 1330.

FIG. 13B illustrates an elevation view of mounting system 1300, including base 1316 for receiving medical sensor 1330.

FIG. 13C illustrates a plan view of mounting system 1300, including base 1316 for receiving medical sensor 1330.

FIG. 14A illustrates a perspective view of a mounting system 1400, including a base 1416 for receiving a medical sensor 1430.

FIG. 14B illustrates an elevation view of mounting system 1400, including base 1416 for receiving medical sensor 1430.

FIG. 14C illustrates an elevation view of mounting system 1400, including base 1416 for receiving medical sensor 1430.

FIG. 14D illustrates a plan view of mounting system 1400, including base 1416 for receiving medical sensor 1430.

FIG. 15A illustrates a perspective view of a mounting system 1500, including a base 1516 for receiving a medical sensor 1530.

FIG. 15B illustrates an elevation view of mounting system 1500, including base 1516 for receiving medical sensor 1530.

FIG. 15C illustrates an elevation view of mounting system 1500, including base 1516 for receiving medical sensor 1530.

FIG. 15D illustrates a plan view of mounting system 1500, including base 1516 for receiving medical sensor 1530.

FIG. 16A illustrates a perspective view of a mounting system 1600, including a base 1616 for receiving a medical sensor 1630.

FIG. 16B illustrates an elevation view of mounting system 1600, including base 1616 for receiving medical sensor 1630.

FIG. 16C illustrates an elevation view of mounting system 1600, including base 1616 for receiving medical sensor 1630.

FIG. 16D illustrates a plan view of mounting system 1600, including base 1616 for receiving medical sensor 1630.

FIG. 17A illustrates a perspective view of a mounting system 1700, including a base 1716 for receiving a medical sensor 1730.

FIG. 17B illustrates an elevation view of mounting system 1700, including base 1716 for receiving medical sensor 1730.

FIG. 17C illustrates a plan view of mounting system 1700, including base 1716 for receiving medical sensor 1730.

### DETAILED DESCRIPTION

The devices 100, 200, 300, 800, 900, 1000, 1100 described herein ensure accurate placement of a medical sensor (e.g., 430, 530, 630, 730) on a patient's chest (e.g., 532, 732). The medical sensor may be an ultrawideband RF sensor positioned on a patient's chest adjacent to the patient's skin to receive reflected backscatter waves from the patient's chest, where an ultrawideband pulse generator generates one or more ultrawideband pulse and transmits the one or more ultrawideband pulse into the patient's chest. The medical sensor may be an ultrawideband RF sensor, such as disclosed in U.S. patent application Ser. No. 17/260,827 filed on Sep. 23, 2021 and U.S. Patent Application No. 63/321,422 filed on Mar. 18, 2022, each of which is incorporated by reference herein in its entirety.

Devices 100, 200, 300, 800, 900, 1000, 1100 are used to ensure proper placement of a base 116, 216, 316, 416, 516, 616, 716, 816, 916, 1016, 1116, 1216 on the patient's chest, after which medical sensor (e.g., 430, 530, 630, 730) may be placed upon the base. The base may be an adhesive base having a protective backing that is removed prior to placement on the patient's chest.

Devices 100, 200, 300, 800, 900, 1000, 1100 may be used to place medical sensor (e.g., 430, 530, 630, 730) in an optimal location 104, 204, 304, 804, 904, 1004, 1104, 1204 on the patient's chest, which may be between the fourth and fifth intercostal space on a patient's right side, at the right midclavicular line 107, 207, 307, 807, 907, 1007, 1107, 1207. This optimal location 104, 204, 304, 804, 904, 1004, 1104, 1204 may be over the patient's right lung. Device 100, 200, 300, 800, 900, 1000, 1100 may be used by laypersons, caregivers, patients, and medical personnel to accurately and consistently position the sensor in its optimal position.

Vertically, the optimal location 104, 204, 304, 804, 904, 1004, 1104, 1204 may be 67% of the distance of a line starting at the sternal notch 102, 202, 302, 802, 902, 1002, 1102, 1202 below the patient's neck, to the xiphoid process 106, 206, 306, 806, 906, 1006, 1106, 1206 at the bottom of the patient's rib cage/sternum.

Device 100, 200, 300, 800, 900, 1000, 1100 includes a sternal notch index portion 110, 210, 310, 810, 1010, 1110 configured to align with the patient's sternal notch 102, 202, 302, 802, 902, 1002, 1102, 1202.

Device 100, 200, 300, 800, 900, 1000, 1100 includes a xiphoid process index portion 114, 814, 1014, 1114, configured to align with the patient's xiphoid process 106, 206, 306, 806, 906, 1006, 1106, 1206.

Horizontally, the optimal location 104, 204, 304, 804, 904, 1004, 1104, 1204 for placement of the medical sensor (e.g., 430, 530, 630, 730) is on the right midclavicular line 107, 207, 307, 807, 907, 1007, 1107, 1207.

The optimal location 104, 204, 304, 804, 904, 1004, 1104, 1204 varies based upon the general size of the patient. Device 100, 200, 300, 800, 900, 1000, 1100 may include a variety of positions relative to the size of the patient, such as small, medium, and large sizes. A medical professional may assist a patient in determining the patient's size (e.g., medium), which the patient or caregiver will then use as a reference when positioning the medical sensor using device 100, 200, 300, 800, 900, 1000, 1100.

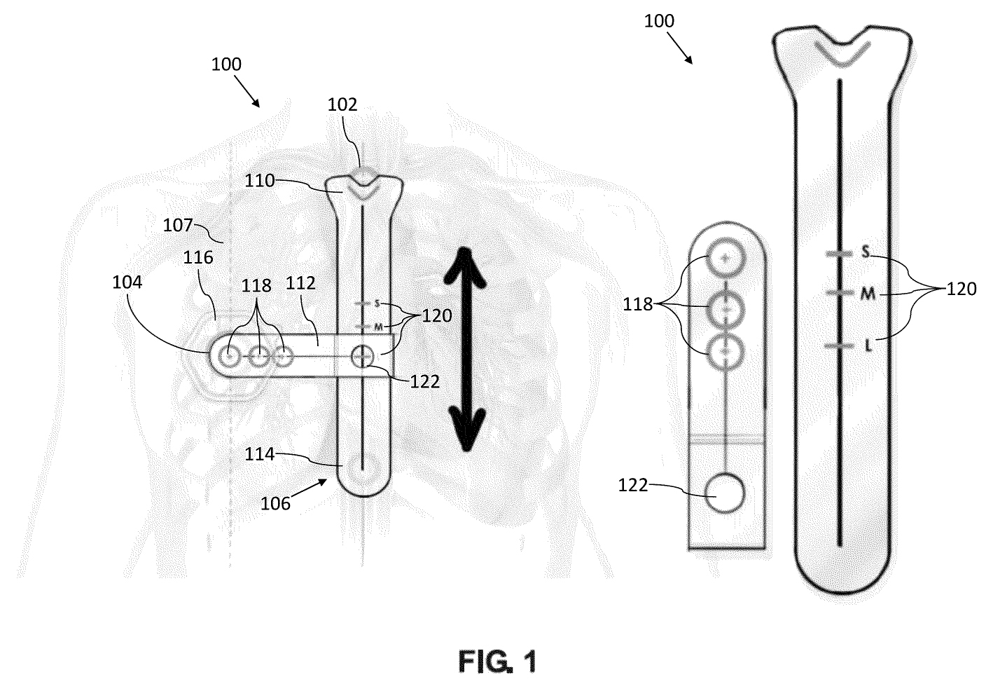

With specific reference to FIG. 1, device 100 includes horizontal patient size indices 118 and vertical patient size indices 120. The sizes may be, for example small, medium, and large. Device 100 includes an arm 112 extending from a vertical portion for the horizontal locating of optimal location 104. Arm 112 may fit over and slide along the vertical portion. Device 100 may include a target aperture 122 for the vertical locating of optimal location 104 on vertical patient size indices 120.

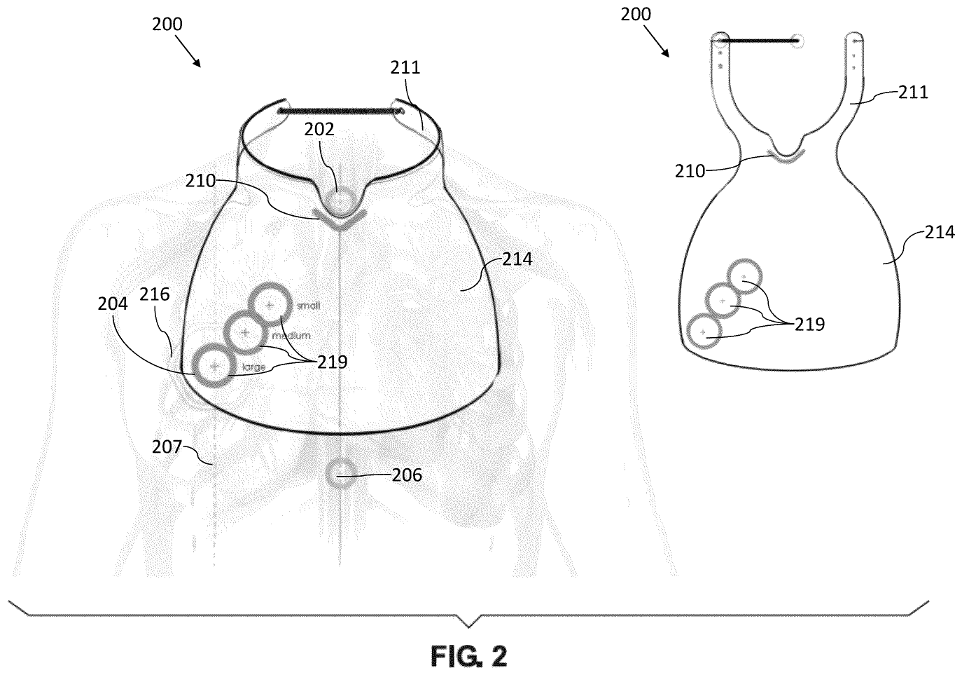

With specific reference to FIG. 2, device 200 includes patient size indices 219. Device 200 includes a collar 211 to extend around a patient's neck. Device 200 includes a bib portion 214 extending downward (inferiorly) from collar 211 toward xiphoid process 206.

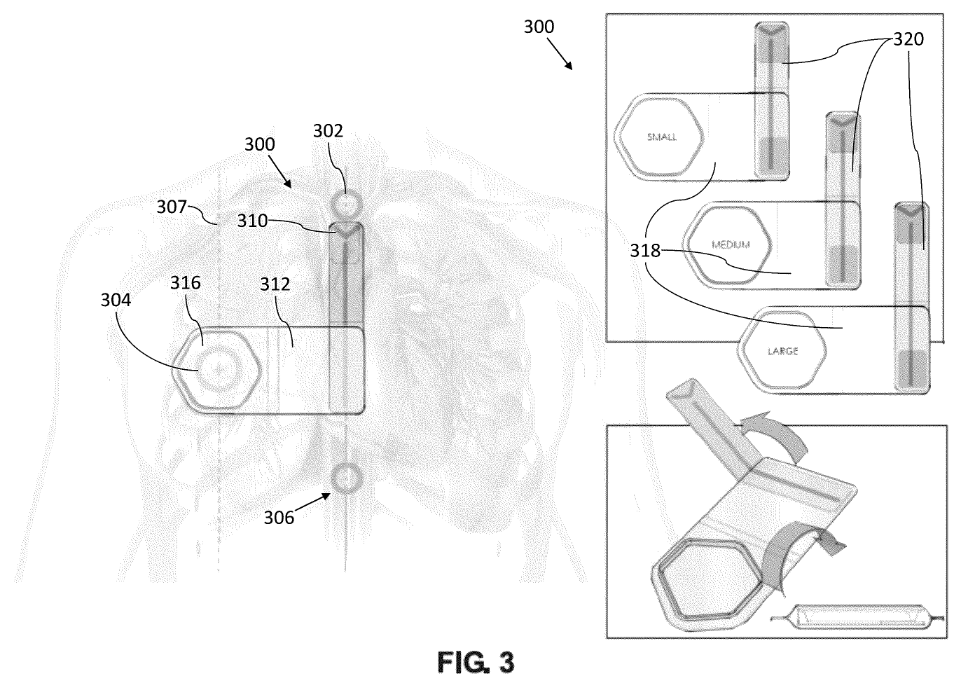

With specific reference to FIG. 3, device 300 includes small, medium, and large devices. The small, medium, and large device 300 include an arm 312 with horizontal patient size elements 318 and vertical patient size elements 320.

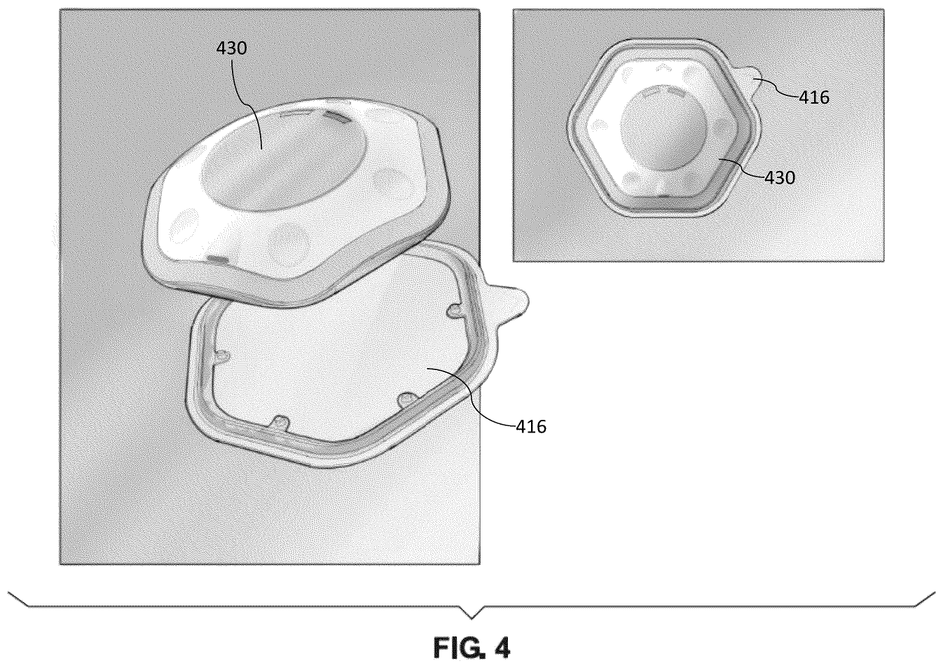

With specific reference to FIG. 4, base 416 is designed to receive a medical sensor 430. Base 416 may include a raised fence within which medical sensor 430 is located after placement of base 416. Sensor 430 may be adhered to base 416.

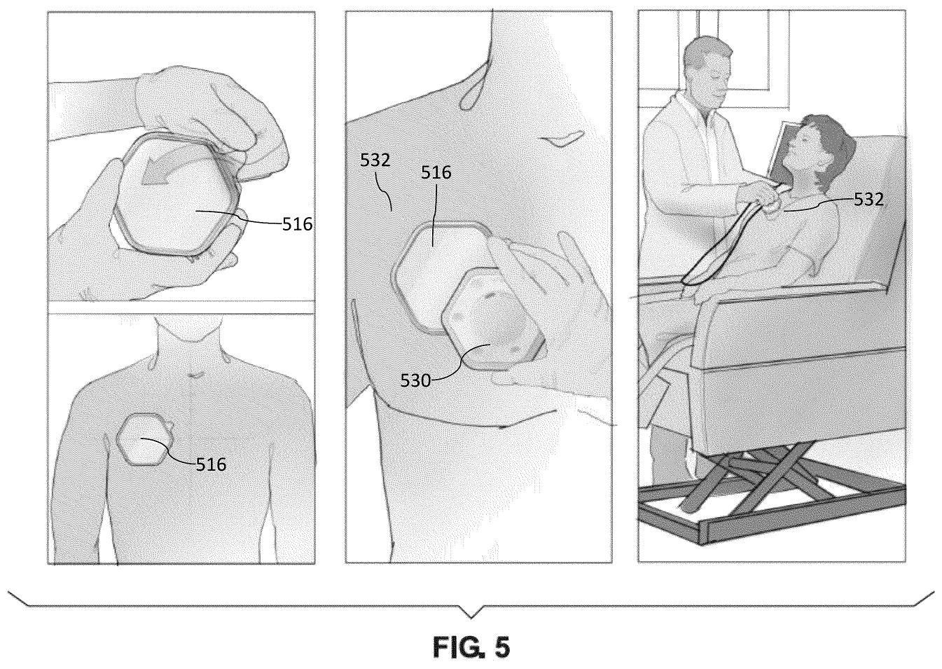

With specific reference to FIG. 5, base 516 includes a protective backer that is removed, after which base 516 is adhered to a patient's chest 532. Medical sensor 530 is subsequently located on base 516. Medical sensor 530 may be adhered to base 516. The patient is oriented in an upward but reclined position while remaining stationary. Sensor 530 is placed for only about one minute of time per sensing operation.

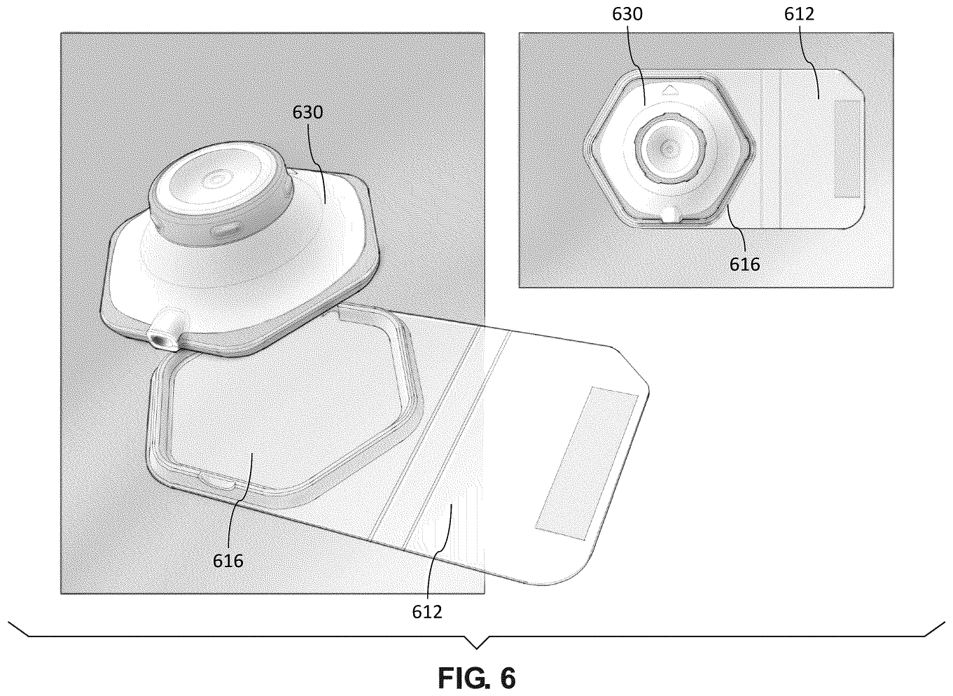

With specific reference to FIG. 6, base 616 is designed to receive a medical sensor 630. Base 616 may include a raised fence within which medical sensor 630 is located after placement of base 616. Sensor 630 may be adhered to base 616.

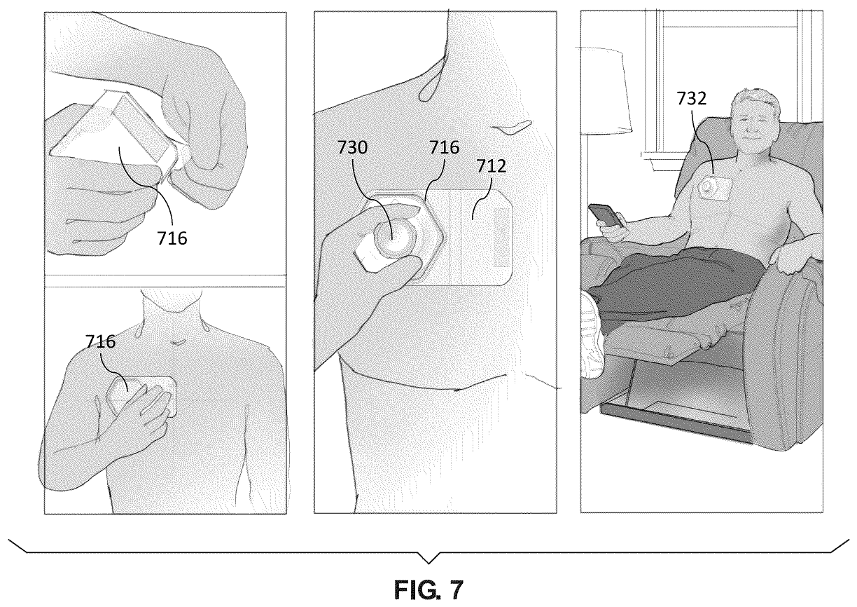

With specific reference to FIG. 7, base 716 includes a protective backer that is removed, after which base 716 is adhered to a patient's chest 732. Medical sensor 730 is subsequently located on base 716. Medical sensor 730 may be adhered to base 716. The patient is oriented in an upward but reclined position while remaining stationary. Sensor 730 is placed for only about one minute of time per sensing operation.

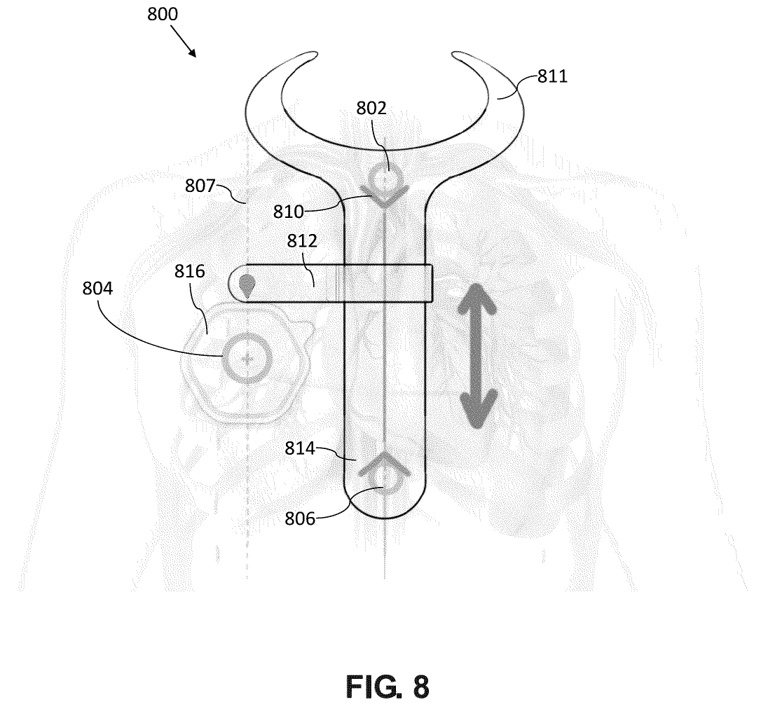

With specific reference to FIG. 8, device 800 includes differently sized horizontal arms 812. Device 800 includes a collar 811 to fit around a patient's neck. Arm 812 can be vertically placed on device 800 based upon vertical patient size indices (not shown).

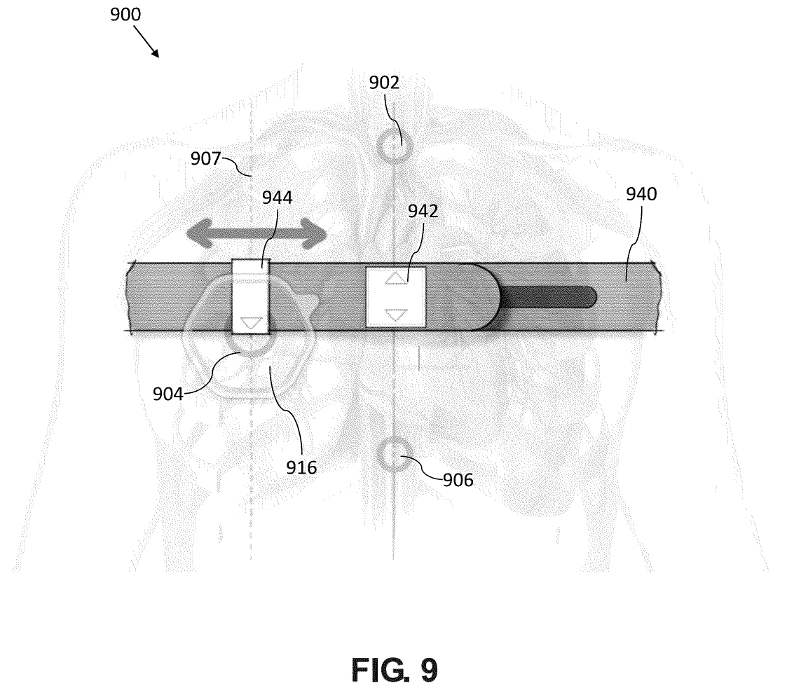

With specific reference to FIG. 9, device 900 includes a band 940 to extend around a patient's torso just under the patient's armpits. Band 940 may include a vertical alignment member 942 and a horizontal alignment member 944.

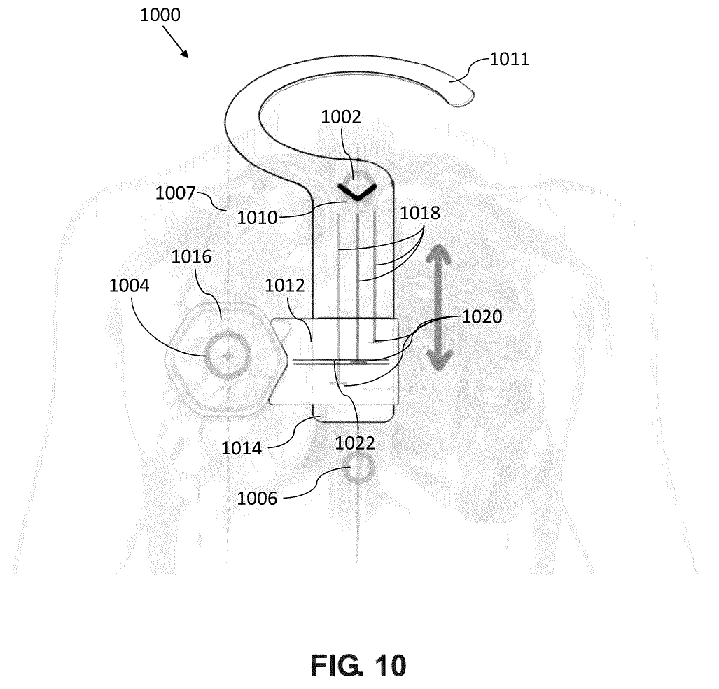

With specific reference to FIG. 10, device 1000 includes a collar 1011, horizontal patient size indices 1018, and vertical patient size indices 1020. The sizes may be, for example small, medium, and large. Device 1000 includes an arm 1012 extending from a vertical portion for the horizontal locating of optimal location 1004. Arm 1012 may fit over and slide along the vertical portion. Device 1000 may include a target aperture 1022 for the horizontal and vertical locating of optimal location 1004 on horizontal and vertical patient size indices 1018 and 1020.

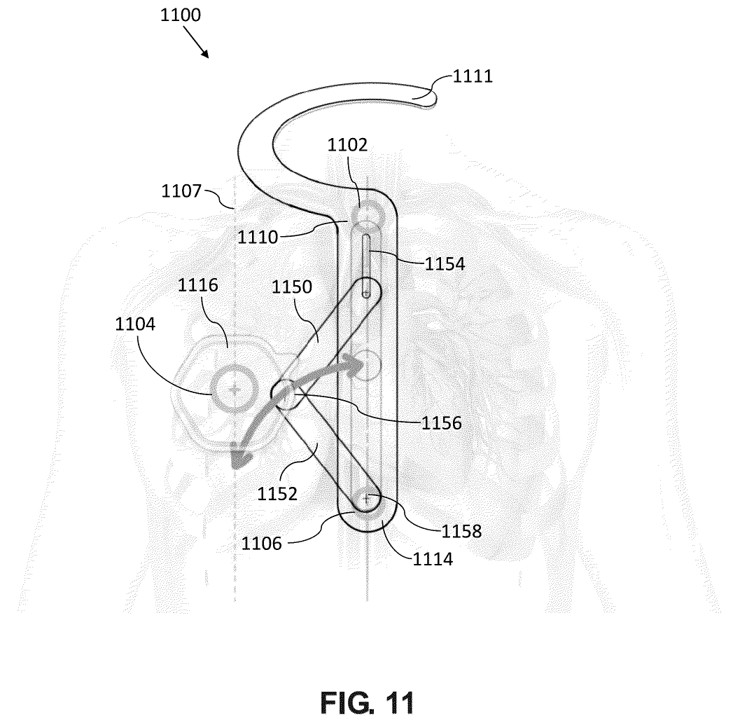

With specific reference to FIG. 11, device 1100 includes a collar 1111. Device 1100 includes a vertical element extending from sternal notch index portion 1110 to xiphoid process index portion 1114, including a vertical slot 1154 receiving an end of a first rotating arm 1150. A second rotating arm 1152 is pivotally connected at a first end to first rotating arm 1150 via a first pivot 1156 and is pivotally connected to the vertical element via a second pivot 1158. Base 1116 is positioned with respect to first pivot 1156. First pivot 1156 moves along a diagonal line oriented superiorly and medially, and inferiorly and laterally. The position of first pivot 1156 may be adjusted based upon the patient's size, referenced by arm 1150's position in vertical slot 1154.

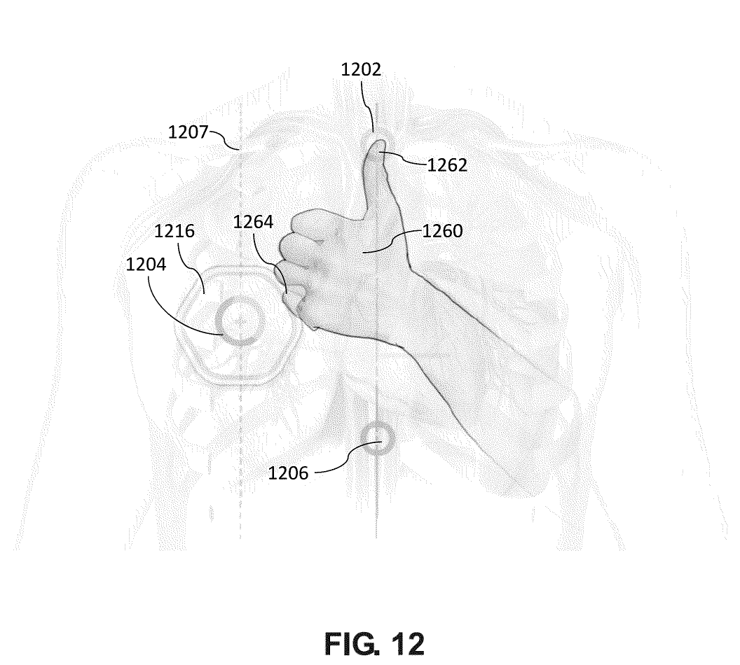

With specific reference to FIG. 12, a patient, caregiver, or medical personnel may position base 1216 by using one's hand 1260 as a placement device. The hand is formed into a fist, with the thumb oriented vertically along the sternum. The tip 1262 of the thumb is placed at the patient's sternal notch 1202. The thumb is oriented along the sternum. The pinky knuckle 1264 is used to identify placement of base 1216.

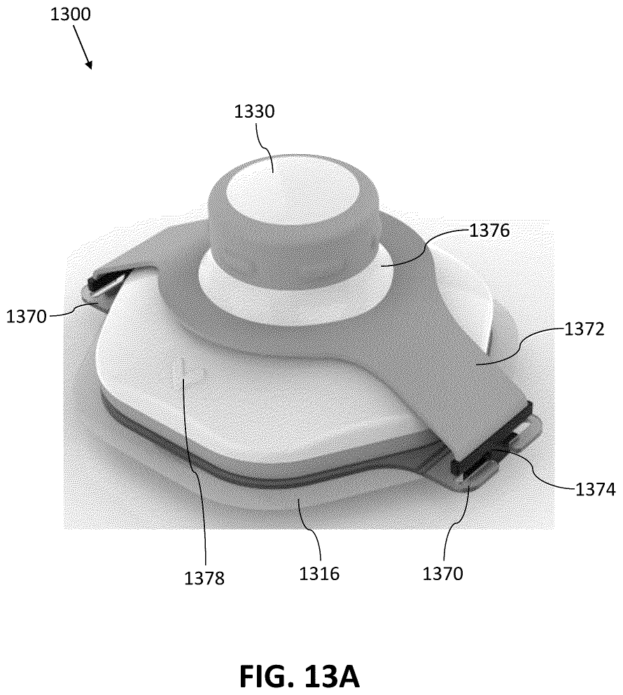

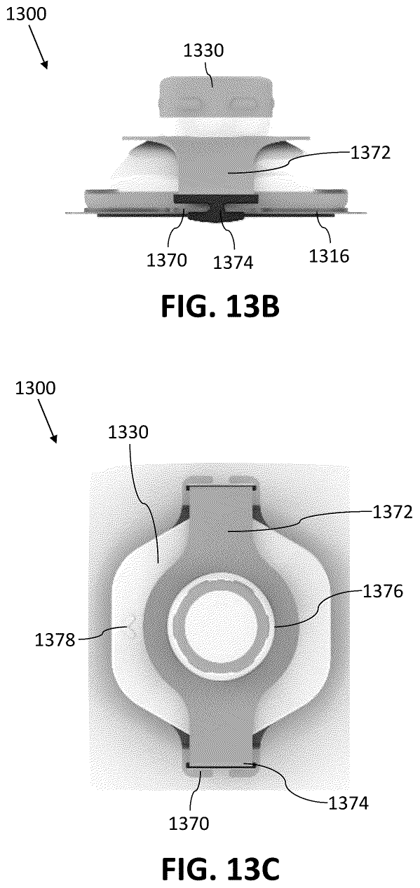

FIGS. 13A-13C illustrate a mounting system 1300 including a base 1316 for receiving a medical sensor 1330. Base 1316 may be attached to a patient's chest as described above with respect to base 116, 216, 316, 416, 516, 616, 716, 816, 916, 1016, 1116, 1216. Base 1316 may be attached to a patient's chest with an adhesive integrated into a lower surface of base 1316. Sensor 1330 may engage base 1316 in a removable manner, such that a patient may apply sensor 1330 to base 1316 for a prescribed amount of time, at prescribed times, to allow sensor 1330 to operate and collect the relevant data.

Base 1316 may include a raised ridge forming a perimeter into which sensor 1330 may be at least partially constrained when engaged with base 1316. Base 1316 may include at least one base connector 1370 extending laterally out of the edge of base 1316. Base 1316 may include two base connectors 1370 oriented at opposite edges of base 1316.

System 1300 may include a flexible retention strap 1372 extending over sensor 1330 and engaging the one or more base connector 1370. Where system 1300 includes two base connectors 1370, strap 1372 extends from a first base connector 1370 to a second base connector 1370.

Strap 1372 may include strap connectors 1374 at distal ends of strap 1372. Strap connectors 1374 are configured to engage base connectors 1370. Strap connectors 1374 may include connector elements designed to hook, engage, snap into, extend around, or extend within base connectors 1370. Strap 1372 may be elastic.

Strap 1372 may include a cutout 1376 designed to permit strap 1372 to fit around sensor 1330. Sensor 1330 may include a raised portion, and cutout 1376 is configured to accommodate the raised portion, increasing engagement between strap 1372 to sensor 1330. Alternatively, or additionally, strap 1372 is fixed to sensor 1330 through any of a variety of mechanisms, including fasteners, adhesive, and the like.

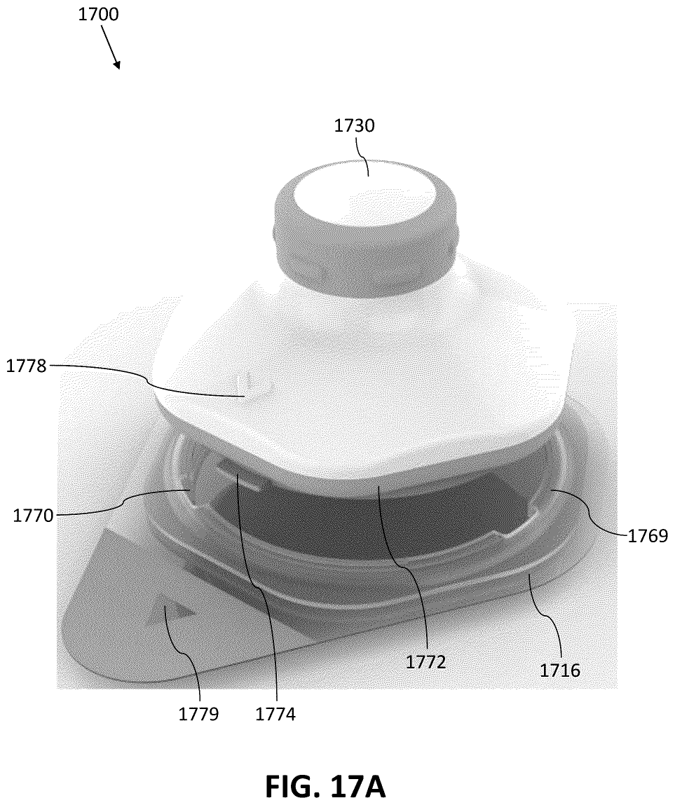

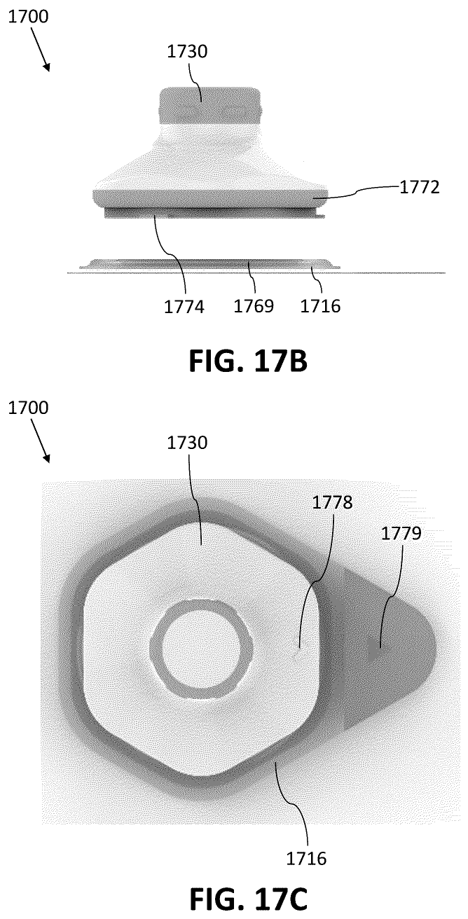

Sensor 1330 may include an orientation element 1378. Orientation element 1378 may be an indicia, mark, element, raised element, symbol, or the like. Orientation element 1378 assists the patient in the proper orientation of sensor 1330. Orientation element 1378 may be, for example, an arrow and patient may be instructed to orient the device so that the arrow points upwardly. Base 1316 may include a corresponding orientation element (as illustrated in FIGS. 17A and 17C as 1779).

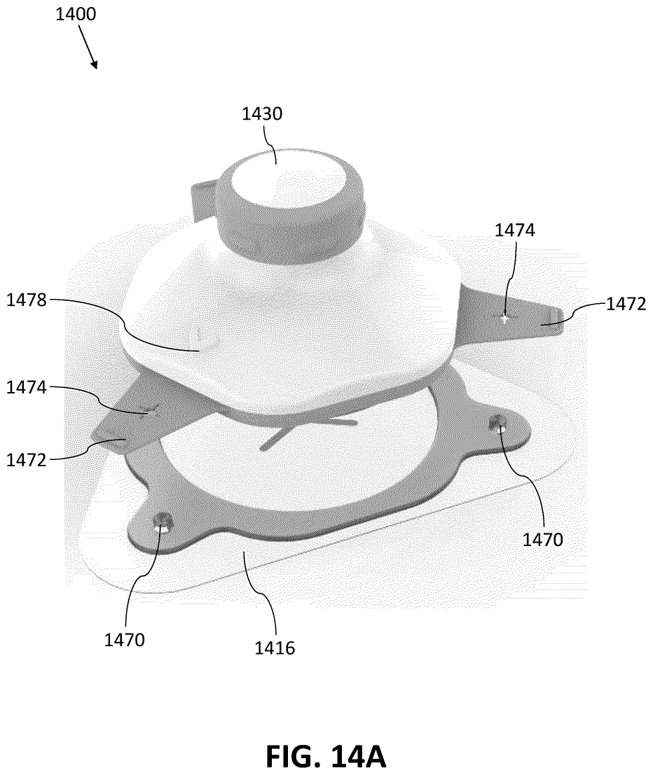

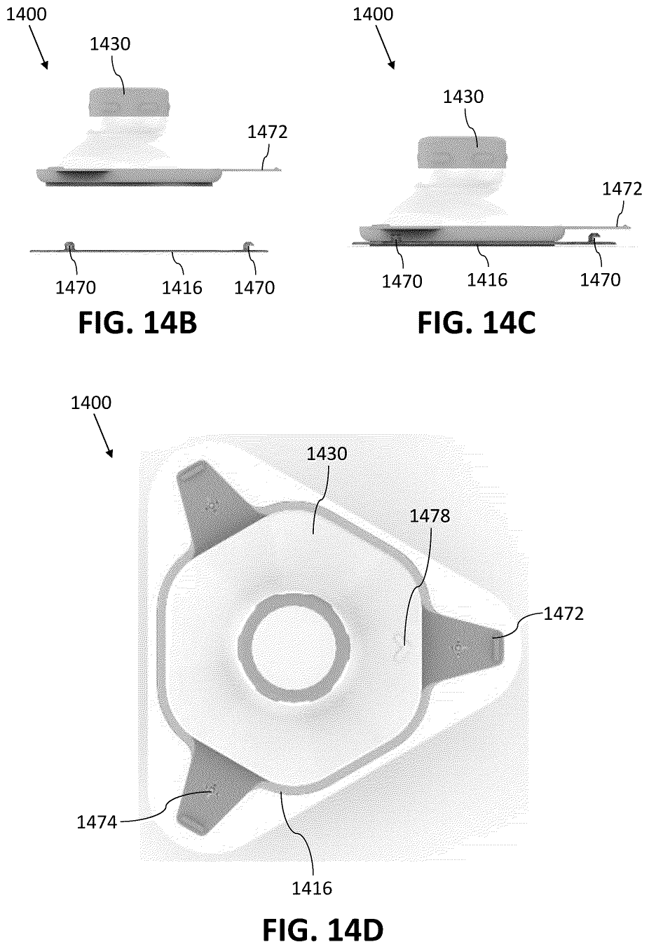

FIGS. 14A-14D illustrate a mounting system 1400, including a base 1416 for receiving a medical sensor 1430. Base 1416 may be attached to a patient's chest as described above with respect to base 116, 216, 316, 416, 516, 616, 716, 816, 916, 1016, 1116, 1216. Base 1416 may be attached to a patient's chest with an adhesive integrated into a lower surface of base 1416. Sensor 1430 may engage base 1416 in a removable manner, such that a patient may apply sensor 1430 to base 1416 for a prescribed amount of time, at prescribed times, to allow sensor 1430 to operate and collect the relevant data.

Base 1416 may include a raised ridge forming a perimeter into which sensor 1430 may be at least partially constrained when engaged with base 1416. Base 1416 may include at least one base connector 1470 extending laterally out of the edge of base 1416. Base 1416 may include three base connectors 1470 oriented in an equally dispersed manner (e.g., at 120 degrees from one another) from base 1416.

System 1400 may include a plurality of flexible retention elements 1472 extending laterally from sensor 1430 and engaging the one or more base connector 1470. Where system 1400 includes three base connectors 1470, three retention elements 1472 extend to the three base connectors 1470.

Base connectors 1470 may be hooked elements hooked away from the center of base 1416. Base connectors 1470 may be pegs with an enlarged head.

Retention elements 1472 may include retention connectors 1474. Retention connectors 1474 are configured to engage base connectors 1470. Retention connectors 1474 may be apertures extending at least partially through, or fully through, retention elements 1472. A user may fit retention connectors 1474 over base connectors 1470, such that base connectors hook into retention connectors 1474. A user may simply unhook retention connectors 1474 and base connectors 1470 when disengaging retention elements 1472.

Retention elements 1472 are fixed to sensor 1430 through any of a variety of mechanisms, including fasteners, adhesive, and the like. Retention elements 1472 may be integrally formed with sensor 1430.

Sensor 1430 may include an orientation element 1478. Orientation element 1478 may be an indicia, mark, element, raised element, symbol, or the like. Orientation element 1478 assists the patient in the proper orientation of sensor 1430. Orientation element 1478 may be, for example, an arrow and patient may be instructed to orient the device so that the arrow points upwardly. Base 1416 may include a corresponding orientation element (as illustrated in FIGS. 17A and 17C as 1779).

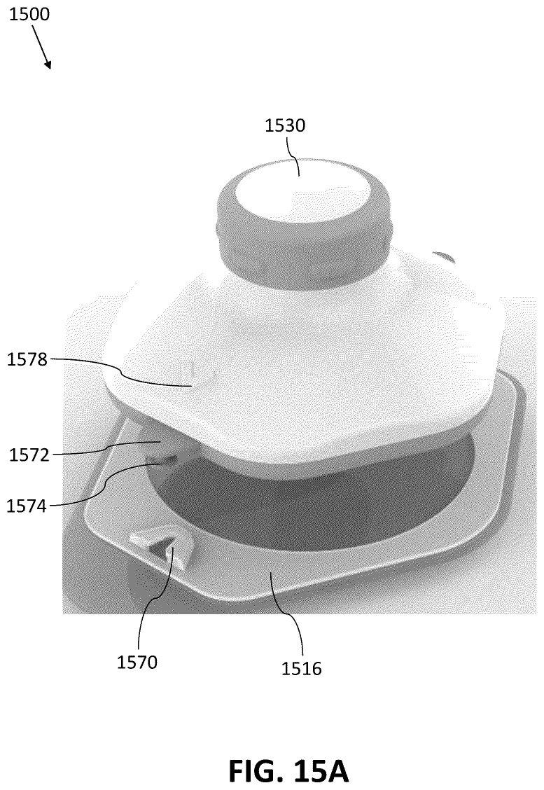

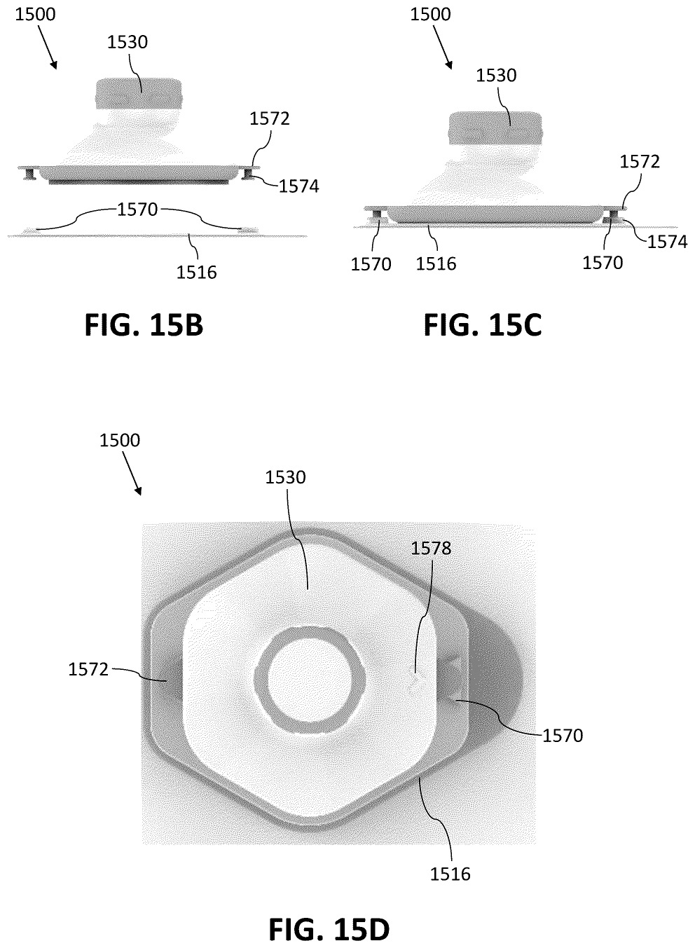

FIGS. 15A-15D illustrate a mounting system 1500, including a base 1516 for receiving a medical sensor 1530. Base 1516 may be attached to a patient's chest as described above with respect to base 116, 216, 316, 416, 516, 616, 716, 816, 916, 1016, 1116, 1216. Base 1516 may be attached to a patient's chest with an adhesive integrated into a lower surface of base 1516. Sensor 150 may engage base 1516 in a removable manner, such that a patient may apply sensor 1530 to base 1516 for a prescribed amount of time, at prescribed times, to allow sensor 1530 to operate and collect the relevant data.

Base 1516 may include at least one base connector 1570 extending upwardly out of base 1516. Base 1516 may include two base connectors 1570 oriented opposite one another from base 1516.

System 1500 may include a plurality of retention elements 1572 extending laterally from sensor 1530 and engaging the one or more base connector 1570. Where system 1500 includes two base connectors 1570, two retention elements 1572 extend to the two base connectors 1570.

Base connectors 1570 may be slots in a V-shape configured to accept an enlarged head of a peg in a lower portion, and the shaft of the peg in an upper portion. Base connectors 1570 may include a detent configured to hold the peg in place upon engagement. Base connectors 1570 face the same direction.

Retention elements 1572 may include retention connectors 1574. Retention connectors 1574 are configured to engage base connectors 1570. Retention connectors 1574 may be pegs extending downwardly from sensor 1530. The pegs may have an enlarged head, and narrower shaft portion, and may be sized and shaped to fit into base connectors 1570. Base connectors 1570 face the same direction, such that retention connectors 1574 maybe be simultaneously slid laterally into the V-shaped base connectors 1570. A detent in base connectors 1570 may keep retention connectors 1574 held in position, and a user may simply slide retention connectors 1574 and base connectors 1570 apart in a direction opposite the engagement direction when disengaging retention elements 1572.

Retention elements 1572 are fixed to sensor 1530 through any of a variety of mechanisms, including fasteners, adhesive, and the like. Retention elements 1572 may be integrally formed with sensor 1530.

Sensor 1530 may include an orientation element 1578. Orientation element 1578 may be an indicia, mark, element, raised element, symbol, or the like. Orientation element 1578 assists the patient in the proper orientation of sensor 1530. Orientation element 1578 may be, for example, an arrow and patient may be instructed to orient the device so that the arrow points upwardly. Base 1516 may include a corresponding orientation element (as illustrated in FIGS. 17A and 17C as 1779).

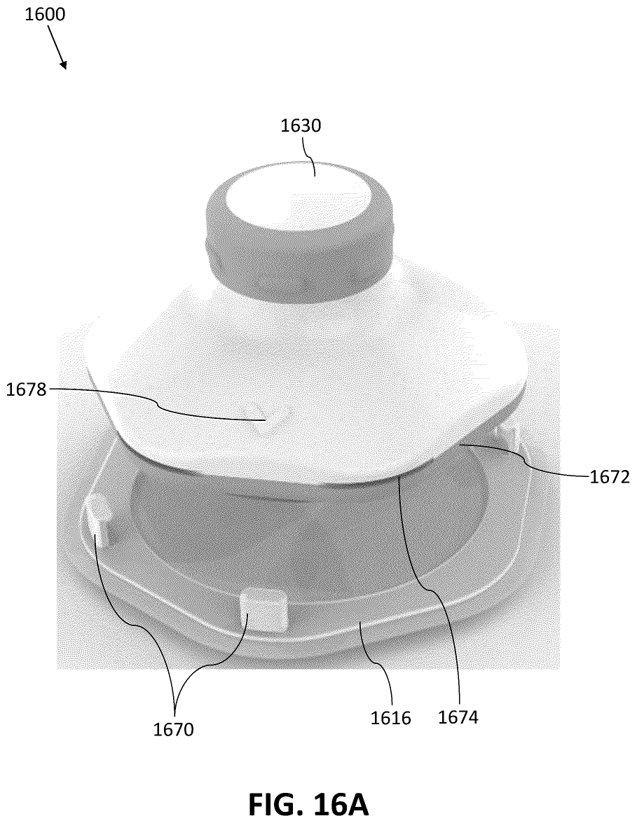

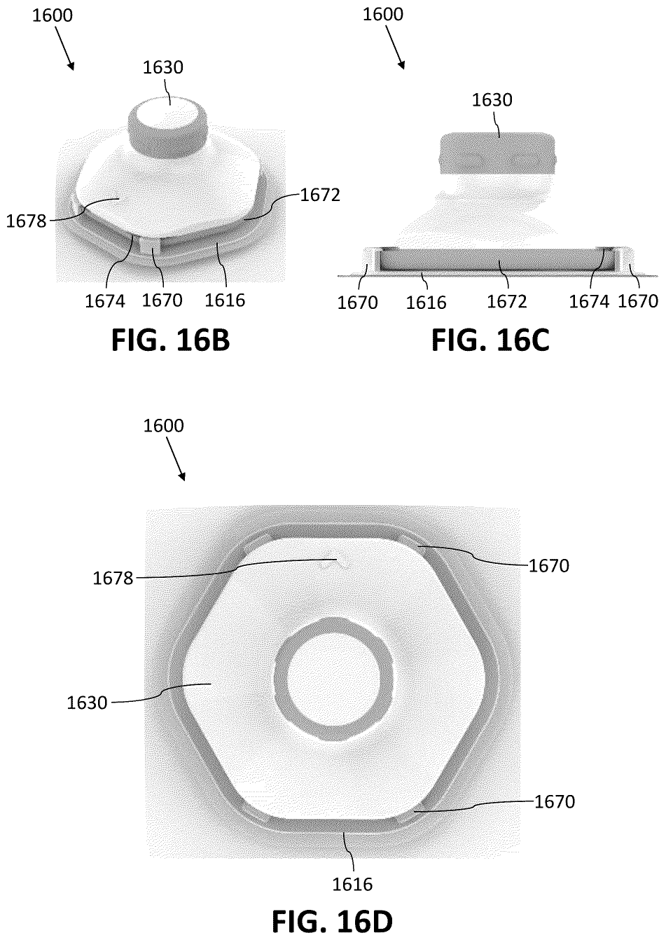

FIGS. 16A-16D illustrate a mounting system 1600, including a base 1616 for receiving a medical sensor 1630. Base 1616 may be attached to a patient's chest as described above with respect to base 116, 216, 316, 416, 516, 616, 716, 816, 916, 1016, 1116, 1216. Base 1616 may be attached to a patient's chest with an adhesive integrated into a lower surface of base 1616. Sensor 1630 may engage base 1616 in a removable manner, such that a patient may apply sensor 1630 to base 1616 for a prescribed amount of time, at prescribed times, to allow sensor 1630 to operate and collect the relevant data.

Base 1616 may include a raised ridge forming a perimeter into which sensor 1630 may be at least partially constrained when engaged with base 1616. Base 1616 may include at least one base connector 1670 extending vertically out of the top of base 1616. Base 1616 may include a plurality of base connectors 1670 oriented about the edge of base 1616.

System 1600 may include a retention element 1672 extending downwardly from sensor 1630 and engaging the one or more base connector 1670.

Base connectors 1670 may be tab elements hooked toward the center of base 1616.

Retention element 1672 may include a plurality of retention slots 1674. Retention slots 1674 are configured to engage base connectors 1670. Retention slots 1674 may be slots extending into retention element 1672 substantially parallel to the bottom of sensor 1630. A user may lower sensor 1630 onto base 1616 in the rotational position illustrated in FIG. 16A, and then rotate sensor 1630 relative to base 1616 to cause the upper tab portion of each base connector 1670 to engage retention slots 1674.

A user may rotate sensor 1630 until the tabs of base connectors 1670 encounter a stop within retention slots 1674, preventing sensor 1630 from rotating further. Sensor 1630 may need to be rotated 15 degrees, 30 degrees, somewhere between 15 degrees and 30 degrees, or the like. Sensor 1630 and/or retention element 1672 may include indicia to be aligned with corresponding indicia on base 1616 to instruct a user how to initially align sensor 1630 with base 1616 (e.g., the alignment illustrated in FIG. 16A), and then a direction to rotate sensor 1630 to cause base connectors 1670 to engage retention slots 1674. FIGS. 16B-16D illustrate sensor 1630 locked into base 1616, with base connectors 1670 engaged with retention slots 1674.

Retention element 1672 is fixed to sensor 1630 through any of a variety of mechanisms, including fasteners, adhesive, and the like. Retention element 1672 may be integrally formed with sensor 1630.

Sensor 1630 may include an orientation element 1678. Orientation element 1678 may be an indicia, mark, element, raised element, symbol, or the like. Orientation element 1678 assists the patient in the proper orientation of sensor 1630. Orientation element 1678 may be, for example, an arrow and patient may be instructed to orient the device so that the arrow points upwardly. Base 1616 may include a corresponding orientation element (as illustrated in FIGS. 17A and 17C as 1779).

FIGS. 17A-17C illustrate a mounting system 1700, including a base 1716 for receiving a medical sensor 1730. Base 1716 may be attached to a patient's chest as described above with respect to base 116, 216, 316, 416, 516, 616, 716, 816, 916, 1016, 1116, 1216. Base 1716 may be attached to a patient's chest with an adhesive integrated into a lower surface of base 1716. Sensor 1730 may engage base 1716 in a removable manner, such that a patient may apply sensor 1730 to base 1716 for a prescribed amount of time, at prescribed times, to allow sensor 1730 to operate and collect the relevant data.

Base 1716 may include a raised ridge 1769 forming a perimeter into which sensor 1730 may be at least partially constrained when engaged with base 1716. Base 1716 may include at least one base connector 1770 extending out of the top of base 1716, including a slot formed in ridge 1769, and a cutout for permitting passage of a retention tab 1774. Base 1716 may include a plurality of base connectors 1770 oriented about the edge of base 1716.

System 1700 may include a retention element 1772 extending downwardly from sensor 1730 and engaging the one or more base connector 1770.

Retention element 1772 may include a plurality of retention tabs 1774. Retention tabs 1774 are configured to engage base connectors 1770. Retention tabs 1774 may be tabs extending from retention element 1772 substantially parallel to the bottom of sensor 1730. A user may lower sensor 1730 onto base 1716 in a rotational position wherein retention tabs 1774 are rotationally aligned with slots cutouts in base connector 1770, and then rotate sensor 1730 relative to base 1716 to cause the slot of each base connector 1770 to engage retention tabs 1774.

A user may rotate sensor 1730 until retention tabs 1774 encounter a stop within the slots of base connectors 1770, preventing sensor 1730 from rotating further. Sensor 1730 may need to be rotated 15 degrees, 30 degrees, somewhere between 15 degrees and 30 degrees, or the like. Sensor 1730 and/or retention element 1772 may include indicia to be aligned with corresponding indicia on base 1716 to instruct a user how to initially align sensor 1730 with base 1716, and then a direction to rotate sensor 1730 to cause base connectors 1770 to engage retention tabs 1774.

Retention element 1772 is fixed to sensor 1730 through any of a variety of mechanisms, including fasteners, adhesive, and the like. Retention element 1772 may be integrally formed with sensor 1730.

Sensor 1730 may include an orientation element 1778. Orientation element 1778 may be an indicia, mark, element, raised element, symbol, or the like. Orientation element 1778 assists the patient in the proper orientation of sensor 1730. Orientation element 1778 may be, for example, an arrow and patient may be instructed to orient the device so that the arrow points upwardly. Base 1716 may include a corresponding orientation element 1779, and a user may orient sensor 1730 so that orientation elements 1778 and 1779 are aligned for proper function.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See Bryan A. Garner, A Dictionary of Modern Legal Usage 624 (2d. Ed. 1995). Also, to the extent that the terms “in” or “into” are used in the specification or the claims, it is intended to additionally mean “on” or “onto.” To the extent that the term “substantially” is used in the specification or the claims, it is intended to take into consideration the degree of precision available in manufacturing. To the extent that the term “selectively” is used in the specification or the claims, it is intended to refer to a condition of a component wherein a user of the apparatus may activate or deactivate the feature or function of the component as is necessary or desired in use of the apparatus. To the extent that the term “operatively connected” is used in the specification or the claims, it is intended to mean that the identified components are connected in a way to perform a designated function. As used in the specification and the claims, the singular forms “a,” “an,” and “the” include the plural. Finally, where the term “about” is used in conjunction with a number, it is intended to include ±10% of the number. In other words, “about 10” may mean from 9 to 11.

As stated above, while the present application has been illustrated by the description of embodiments and aspects thereof, and while the embodiments and aspects have been described in considerable detail, it is not the intention of the applicants to restrict or in any way limit the scope of the appended claims to such detail. Additional advantages and modifications will readily appear to those skilled in the art, having the benefit of the present application. Therefore, the application, in its broader aspects, is not limited to the specific details, illustrative examples shown, or any apparatus referred to. Departures may be made from such details, examples, and apparatuses without departing from the spirit or scope of the general inventive concept.

What is claimed is:

1. A device for determining a precise placement of a medical sensor on a patient's chest, comprising:
a vertical portion including a sternal notice index portion for alignment with the patient's sternal notch, wherein the sternal notch index portion extends along a portion of the patient's sternum,
wherein the vertical portion includes vertical patient size indices; and
an arm extending from the vertical portion across the patient's midclavicular line, wherein the arm is oriented at a right angle to the vertical portion,
wherein the arm includes horizontal patient size indices.

2. The device of claim 1, wherein the vertical portion further comprises a xiphoid process index portion for alignment with the patient's xiphoid process.

3. The device of claim 1, wherein the vertical patient size indices include three vertical patient size indices labeled small, medium, and large.

4. The device of claim 1, wherein the horizontal patient size indices include three horizontal patient size indices labeled small, medium, and large.

5. The device of claim 1, wherein the device includes a target aperture within the arm to view the vertical patient size indices.

6. The device of claim 1, wherein the arm extends from the vertical portion across the patient's right midclavicular line.

7. The device of claim 1, wherein the device is used to identify an optimal location for placement of a base to receive the medical sensor.

8. A system for mounting a base and a medical sensor on a patient's body, comprising:
a medical sensor including a retention element,
wherein the retention element includes at least one retention slot; and
a base including at least one base connector having an upper tab portion,
wherein the upper tab portion of the at least one base connector engages the at least one retention slot.

9. The system of claim 8, wherein the base includes an adhesive integrated into a lower surface of the base.

10. The system of claim 8, wherein the sensor is placed onto the base and rotated relative to the base to cause the upper tab portion of the at least one base connector to engage the at least one retention slot.

11. The system of claim 10, wherein the at least one retention slot includes a stop to stop rotation of the sensor relative to the base at a desired position.

12. The system of claim 10, wherein the sensor is rotated between 15 degrees and 30 degrees to engage the base.

13. The system of claim 8, wherein the sensor further comprises an orientation element to indicate proper orientation of the sensor relative to the patient's body.

14. A system for mounting a base and a medical sensor on a patient's body, comprising:
a medical sensor including a retention element,
wherein the retention element includes at least one retention tab; and
a base including at least one base connector having a slot and a cutout,
wherein the slot of the at least one base connector engages the at least one retention tab.

15. The system of claim 14, wherein the base includes an adhesive integrated into a lower surface of the base.

16. The system of claim 14, wherein the sensor is placed onto the base and rotated relative to the base to cause the slot of the at least one base connector to engage the at least one retention tab.

17. The system of claim 16 wherein the slot of the base connector includes a stop to stop rotation of the sensor relative to the base at a desired position.

18. The system of claim 16, wherein the sensor is rotated between 15 degrees and 30 degrees to engage the base.

19. The system of claim 14, wherein the sensor further comprises an orientation element to indicate proper orientation of the sensor relative to the patient's body.

20. The system of claim 19, wherein the base includes an orientation element to indicate proper orientation of the sensor relative to the base.
